(19) United States
(12) Patent Application Publication
(10) Pub. No.: US 2023/0355586 A1
(43) Pub. Date: Nov. 9, 2023

(54) Application of Valdecoxib in preparation of medications for preventing and treating glaucoma

(71) Applicant: Central South University, Changsha (CN)

(72) Inventors: Zhaolin Gao, Changsha (CN); Jufang Huang, Changsha (CN)

(21) Appl. No.: 18/215,176

(22) Filed: Jun. 28, 2023

(30) Foreign Application Priority Data
Jun. 28, 2022 (CN) 202210739448.X

(51) Int. Cl. A61K 31/415; A61P 27/06

(52) U.S. Cl. CPC A61K 31/415; A61P 27/06

(57) ABSTRACT

A preparation method of a medication associated with a PERK-ATF4-CHOP signaling pathway for preventing and treating glaucoma includes a step of administering VAL (Valdecoxib). The VAL is a regulating agent for the PERK-ATF4-CHOP signaling pathway. Through regulating the PERK-ATF4-CHOP signaling pathway, the VAL inhibits ERS (endoplasmic reticulum stress), so as to prevent and treat glaucoma. A medication composition for preventing and treating glaucoma includes VAL and a pharmaceutically acceptable carrier.

### CROSS REFERENCE OF RELATED APPLICATION

The present invention claims priority under 35 U.S.C. 119(a-d) to CN 202210739448.X, filed Jun. 28, 2022.

### BACKGROUND OF THE PRESENT INVENTION

### Field of Invention

The present invention relates to the field of biomedical and pharmaceutical technology, and more particularly to an application of valdecoxib in preparation of medications for preventing and treating glaucoma.

### Description of Related Arts

IR (ischemia-reperfusion) is a pathological process involving a variety of diseases and occurs in multiple organs. Organ injury occurs when an organ undergoes the IR. When retinal IR occurs, there may be a variety of eye diseases that affect vision, such as acute glaucoma, diabetic retinopathy, ischemic optic neuropathy, and retinal vasculopathy. Glaucoma is the leading cause of irreversible blindness worldwide, characterized by the progressive loss of the visual field and retinal ganglion cells, as well as optic nerve damage. Approximately 57.5 million people worldwide are affected by POAG (primary open angle glaucoma), with a prevalence of 2.2%. Tham et al. predicted that the number of glaucoma patients aged 40-80 years would increase from 76 million in 2020 to 111.8 million in 2040. Due to the progressive and irreversible nature of glaucoma injury, patients will gradually lose sight and even blindness. At present, there is no effective way to cure the disease and reverse glaucoma injury. Therefore, it is important to find new treatments for glaucoma.

The pathogenesis of glaucoma is complex. Studies in vivo and in vitro on glaucoma models have shown that IR injury, oxidative stress, inflammation, glutamate excitotoxicity, impaired microcirculation, and dysfunction of immune response may be associated with glaucoma. Acute high intraocular pressure (HIOP) model and acute optic nerve injury are common models for simulating the pathological process of glaucoma IR injury. Studies have shown that increased ERS (endoplasmic reticulum stress) proteins in RGCs are observed in animal models of chronic glaucoma and acute optic nerve injury. The ER is the main intracellular organelle responsible for protein synthesis, including protein folding, maturation, and transport. It is affected by environmental changes. Different pathological and physiological conditions, nutritional deficiencies, changes in REDOX status, and viral infections all affect the ability of the ER to facilitate protein folding, leading to the accumulation of unfolded or misfolded proteins in the ER lumen, thereby increasing the ERS.

VAL (valdecoxib) is a selective COX-2 inhibitor, which is widely used clinically for the treatment of knee and hip osteoarthritis, rheumatoid arthritis, dysmenorrhea analgesia, and postoperative analgesia for hip replacement, foot orthopedics and oral surgery. However, there is still a lack of research on the role of VAL in glaucoma injury.

### SUMMARY OF THE PRESENT INVENTION

In view of the above deficiencies, the present invention provides the application of VAL (valdecoxib) in the preparation of medications for preventing and treating glaucoma. The present invention discloses the application of VAL in the preparation of medications associated with the PERK-ATF4-CHOP signaling pathway. The VAL is used as a regulating agent for the PERK-ATF4-CHOP signaling pathway to prevent and treat glaucoma. The VAL disclosed in the present invention is able to inhibit ERS (endoplasmic reticulum stress) by regulating the PERK-ATF4-CHOP signaling pathway, so as to realize the prevention and treatment of glaucoma, which is of great significance for the clinical treatment of glaucoma and is able to be used for the development of related medications.

To achieve the above object, the present invention provides a preparation method of a medication associated with a PERK-ATF4-CHOP signaling pathway for preventing and treating glaucoma, wherein the preparation method comprises a step of administering VAL (Valdecoxib).

Preferably, the VAL is a regulating agent for the PERK-ATF4-CHOP signaling pathway to prevent and treat glaucoma.

Preferably, the VAL is able to inhibit ERS (endoplasmic reticulum stress) by regulating the PERK-ATF4-CHOP signaling pathway, so as to prevent and treat glaucoma.

Also, based on the same inventive concept, the present invention provides an medication composition for preventing and treating glaucoma, wherein the medication composition comprises VAL.

Preferably, the medication composition further comprises a pharmaceutically acceptable carrier.

The present invention has some beneficial effects as follows.

The present invention is the first to use the VAL to prevent and treat glaucoma.

The VAL increases the cell viability in the OGD/R (oxygen-glucose deprivation and reoxygenation) model and reverses the damage induced by acute high intraocular pressure model. The VAL is able to reduce the loss of RGCs (retinal ganglion cells) which are mediated by IRI (ischemia-reperfusion injury) by inhibiting the apoptosis of RGCs. The VAL inhibits the apoptosis of R28 cells by decreasing the ERS (endoplasmic reticulum stress) mediated by the PERK-ATF4-CHOP pathway. The VAL protects the retina from the IRI-mediated apoptosis by decreasing the PERK-ATF4-CHOP pathway-mediated ER stress. CCT020312, the agonist of PERK, reverses the anti-apoptosis effect of VAL by activating the PERK-ATF4-CHOP pathway to activate the ERS. Therefore, the VAL prevents glaucoma injury by inhibiting the PERK-ATF4-CHOP pathway to inhibit the ERS-induced apoptosis. The VAL is expected to become a very promising medication for glaucoma treatment in the future.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A shows R28 cells in the control group and at multiple time points for the OGD/R model groups, stained with PI (red) and Hoechest (blue), in which scale bar=50 μm.

FIG. 1B shows the proportion of PI-positive R28 cells in the control group and OGD/R 0 h, 2 h, 4 h, 8 h and 12 h groups, in which data are presented as the mean±SD of three independent experiments, ****p<0.0001, **p<0.01 vs. control group.

FIG. 1C shows that CCK-8 is used to detect R28 cells' survival rates in the control group, OGD/R group and OGD/R groups pretreated with valdecoxib of different concentrations, in which the data show a significant increase in the cell survival rate observed at the concentrations of 1 and 5 μm VAL compared to the OGD/R group, data are presented as the mean±SD of three independent experiments, ***p<0.001 vs. control group, ##p<0.01, ###p<0.001 vs. OGD/R group.

FIG. 1D shows R28 cells in the control, OGD/R and OGD/R+VAL groups, stained with PI (red) and Hoechest (blue), in which scale bar=50 μm.

FIG. 1E shows the apoptosis of R28 cells obtained by flow cytometry in the control group.

FIG. 1F shows the apoptosis of R28 cells obtained by flow cytometry in the OGD/R group.

FIG. 1G shows the apoptosis of R28 cells obtained by flow cytometry in the OGD/R+VAL group.

FIG. 1H is a statistical graph showing the apoptosis rate by flow cytometry in the control group, the OGD/R group and the OGD/R+VAL group, in which the untreated control group is assigned a survival rate of 100%, data are presented as the mean±SD of three independent experiments, **p<0.01 vs. control group, #p<0.05 vs. OGD/R group.

FIG. 2A shows representative images of vertical sections obtained from retinas in the control, 1d-I/R, 3ds-I/R and 7ds-I/R groups, stained with hematoxylin (blue) and eosin (red), in which scale bar=50 μm.

FIG. 2B shows quantification of the mean total thickness of the retina in the control and 1-, 3- and 7-day post-I/R groups, in which the retinas of the 3ds-I/R and 7ds-I/R groups are significantly thinner compared to those of the control group, the data are presented as the mean±SD of three independent experiments, each group is composed of five rats, **p<0.01, *p<0.05 vs. sham group.

FIG. 2C shows representative images of vertical sections obtained from retinas in the control, 3ds-I/R, 3ds-I/R+DMSO and 3ds-I/R+VAL groups, stained with hematoxylin (blue) and eosin (red), in which scale bar=50 μm.

FIG. 2D shows images obtained from retinas in the control, FR, I/R+DMSO and I/R+VAL groups, stained with DAPI (blue), RBPMS (green) and TUNEL (red), in which scale bar=25 μm, RBPMS is used to label RGCs, and TUNEL is used to label apoptotic cells.

FIG. 3A shows Western blot analysis of p-PERK, ATF4, GRP78, CHOP, cleaved caspase 3, bax and bcl-2 levels in the control, OGD/R, OGD/R+DMSO and OGD/R+VAL groups.

FIG. 3B shows quantification of the expression level of p-PERK in the control, OGD/R, OGD/R+DMSO and OGD/R+VAL groups using the densitometric analyses of Western blot, in which the bar charts show the quantitative data (normalized by β-tubulin) for each protein relative to the control group (assigned a value of 1), data are represented as the mean±SD of three independent experiments, one-way ANOVA is used, *p<0.05, **p<0.01, ***p<0.001 vs. control group, #p<0.05, ##p<0.01, ###p<0.001 vs. OGD/R group, $p<0.05, $$p<0.01, $$$p<0.001 vs. OGD/R+DMSO group.

FIG. 3C shows quantification of the expression level of ATF4 in the control, OGD/R, OGD/R+DMSO and OGD/R+VAL groups using the densitometric analyses of western blot, in which the bar charts show the quantitative data (normalized by β-tubulin) for each protein relative to the control group (assigned a value of 1), data are represented as the mean±SD of three independent experiments, one-way ANOVA is used, *p<0.05, **p<0.01, ***p<0.001 vs. control group, #p<0.05, ##p<0.01, ###p<0.001 vs. OGD/R group, $p<0.05, $$p<0.01, $$$p<0.001 vs. OGD/R+DMSO group.

FIG. 3D shows quantification of the expression level of CHOP in the control, OGD/R, OGD/R+DMSO and OGD/R+VAL groups using the densitometric analyses of western blot, in which the bar charts show the quantitative data (normalized by β-tubulin) for each protein relative to the control group (assigned a value of 1), data are represented as the mean±SD of three independent experiments, one-way ANOVA is used, *p<0.05, **p<0.01, ***p<0.001 vs. control group, #p<0.05, ##p<0.01, ###p<0.001 vs. OGD/R group, $p<0.05, $$p<0.01, $$$p<0.001 vs. OGD/R+DMSO group.

FIG. 3E shows quantification of the expression level of GRP78 in the control, OGD/R, OGD/R+DMSO and OGD/R+VAL groups using the densitometric analyses of western blot, in which the bar charts show the quantitative data (normalized by β-tubulin) for each protein relative to the control group (assigned a value of 1), data are represented as the mean±SD of three independent experiments, one-way ANOVA is used, *p<0.05, **p<0.01, ***p<0.001 vs. control group. #p<0.05, ##p<0.01, ###p<0.001 vs. OGD/R group, $p<0.05, $$p<0.01, $$$p<0.001 vs. OGD/R+DMSO group.

FIG. 3F shows quantification of the expression level of c-caspase 3 in the control, OGD/R, OGD/R+DMSO and OGD/R+VAL groups using the densitometric analyses of western blot, the bar charts show the quantitative data (normalized by β-tubulin) for each protein relative to the control group (assigned a value of 1), data are represented as the mean±SD of three independent experiments, one-way ANOVA is used, *p<0.05, **p<0.01, ***p<0.001 vs. control group, #p<0.05, ##p<0.01, ###p<0.001 vs. OGD/R group, $p<0.05, $$p<0.01, $$$p<0.001 vs. OGD/R+DMSO group.

FIG. 3G shows quantification of the expression level of bax in the control, OGD/R, OGD/R+DMSO and OGD/R+VAL groups using the densitometric analyses of western blot, in which the bar charts show the quantitative data (normalized by β-tubulin) for each protein relative to the control group (assigned a value of 1), data are represented as the mean±SD of three independent experiments, one-way ANOVA is used, *p<0.05, **p<0.01, ***p<0.001 vs. control group, #p<0.05, ##p<0.01, ###p<0.001 vs. OGD/R group, $p<0.05, $$p<0.01, $$$p<0.001 vs. OGD/R+DMSO group.

FIG. 3H shows quantification of the expression level of bcl-2 in the control, OGD/R, OGD/R+DMSO and OGD/R+VAL groups using the densitometric analyses of western blot, in which the bar charts show the quantitative data (normalized by β-tubulin) for each protein relative to the control group (assigned a value of 1), data are represented as the mean±SD of three independent experiments, one-way ANOVA is used, *p<0.05, **p<0.01, ***p<0.001 vs. control group, #p<0.05, ##p<0.01, ###p<0.001 vs. OGD/R group, $p<0.05, $$p<0.01, $$$p<0.001 vs. OGD/R+DMSO group.

FIG. 4A shows Western blot analysis of p-PERK, ATF4, GRP78, CHOP, cleaved caspase 3, bax and bcl-2 levels in the control, I/R, I/R+DMSO and I/R+VAL groups, in which β-tubulin serves as the loading control.

FIG. 4B shows quantification of the expression level of p-PERK in the control, FR, I/R+DMSO and I/R+VAL groups using the densitometric analyses of western blot, in which the bar charts show the quantitative data (normalized by β-tubulin) for each protein relative to the sham group (assigned a value of 1), data are represented as the mean±SD of three independent experiments, each group is composed of five rats, one-way ANOVA is used, *p<0.05, **p<0.01, ***p<0.001 vs. sham group, ##p<0.01, ###p<0.001 vs. I/R group, $$p<0.01, $$$p<0.001 vs. I/R+DMSO group.

FIG. 4C shows quantification of the expression level of ATF4 in the control, FR, I/R+DMSO and I/R+VAL groups using the densitometric analyses of western blot, in which the bar charts show the quantitative data (normalized by β-tubulin) for each protein relative to the sham group (assigned a value of 1), data are represented as the mean±SD of three independent experiments, each group is composed of five rats, one-way ANOVA is used, *p<0.05, **p<0.01, ***p<0.001 vs. sham group, ##p<0.01, ###p<0.001 vs. I/R group, $$p<0.01, $$$p<0.001 vs. I/R+DMSO group.

FIG. 4D shows quantification of the expression level of CHOP in the control, FR, I/R+DMSO and I/R+VAL groups using the densitometric analyses of western blot, in which the bar charts show the quantitative data (normalized by β-tubulin) for each protein relative to the sham group (assigned a value of 1), data are represented as the mean±SD of three independent experiments, each group is composed of five rats, one-way ANOVA is used, *p<0.05, **p<0.01, ***p<0.001 vs. sham group, ##p<0.01, ###p<0.001 vs. I/R group, $$p<0.01, $$$p<0.001 vs. I/R+DMSO group.

FIG. 4E shows quantification of the expression level of GRP78 in the control, FR, I/R+DMSO and I/R+VAL groups using the densitometric analyses of western blot, in which the bar charts show the quantitative data (normalized by β-tubulin) for each protein relative to the sham group (assigned a value of 1), data are represented as the mean±SD of three independent experiments, each group is composed of five rats, one-way ANOVA is used, *p<0.05, **p<0.01, ***p<0.001 vs. sham group, ##p<0.01, ###p<0.001 vs. I/R group, $$p<0.01, $$$p<0.001 vs. I/R+DMSO group.

FIG. 4F shows quantification of the expression level of c-caspase 3 in the control, FR, I/R+DMSO and I/R+VAL groups using the densitometric analyses of western blot, in which the bar charts show the quantitative data (normalized by β-tubulin) for each protein relative to the sham group (assigned a value of 1), data are represented as the mean±SD of three independent experiments, each group is composed of five rats, one-way ANOVA is used, *p<0.05, **p<0.01, ***p<0.001 vs. sham group, ##p<0.01, ###p<0.001 vs. I/R group, $$p<0.01, $$$p<0.001 vs. I/R+DMSO group.

FIG. 4G shows quantification of the expression level of bax in the control, I/R, I/R+DMSO and I/R+VAL groups using the densitometric analyses of western blot, in which the bar charts show the quantitative data (normalized by β-tubulin) for each protein relative to the sham group (assigned a value of 1), data are represented as the mean±SD of three independent experiments, each group is composed of five rats, one-way ANOVA is used, *p<0.05, **p<0.01, ***p<0.001 vs. sham group, ##p<0.01, ###p<0.001 vs. I/R group, $$p<0.01, $$$p<0.001 vs. I/R+DMSO group.

FIG. 4H shows quantification of the expression level of bcl-2 in the control, FR, I/R+DMSO and I/R+VAL groups using the densitometric analyses of western blot, the bar charts show the quantitative data (normalized by β-tubulin) for each protein relative to the sham group (assigned a value of 1), data are represented as the mean±SD of three independent experiments, each group is composed of five rats, one-way ANOVA is used, *p<0.05, **p<0.01, ***p<0.001 vs. sham group, ##p<0.01, ###p<0.001 vs. I/R group, $$p<0.01, $$$p<0.001 vs. I/R+DMSO group.

FIG. 4I shows representative fluorescence images of p-PERK staining (scale bar=50 μm), in which immunostaining is executed using a primary antibody against p-PERK (green), and the nucleus (blue) is marked by DAPI.

FIG. 4J shows representative fluorescence images of cleaved caspase 3 staining (scale bar=50 μm), in which immunostaining is executed using a primary antibody against cleaved caspase 3 (green), and the nucleus (blue) is marked by DAPI.

FIG. 4K shows representative fluorescence images of GRP78 staining (scale bar=50 μm), in which immunostaining is executed using a primary antibody against GRP78 (green), and the nucleus (blue) is marked by DAPI.

FIG. 5A shows Western blot analysis of p-PERK, ATF4, GRP78, CHOP, cleaved caspase 3, bax and bcl-2 levels in the control, OGD/R+DMSO, OGD/R+VAL, OGD/R+VAL+CCT and OGD/R+DMSO+CCT groups, in which β-tubulin serves as the loading control.

FIG. 5B shows quantification of the expression level of p-PERK in the control, OGD/R+DMSO, OGD/R+VAL, OGD/R+VAL+CCT and OGD/R+DMSO+CCT groups using the densitometric analyses of western blot, in which the bar charts show the quantitative data (normalized by β-tubulin) for each protein relative to the control group (assigned a value of 1), data are represented as the mean±SD of three independent experiments, one-way ANOVA is used, *p<0.05, **p<0.01, ***p<0.001 vs. control group, #p<0.05, ##p<0.01, ###p<0.001 vs. OGD/R+DMSO group, $p<0.05, $$p<0.01, $$$p<0.001 vs. OGD/R+VAL group.

FIG. 5C shows quantification of the expression level of ATF4 in the control, OGD/R+DMSO, OGD/R+VAL, OGD/R+VAL+CCT and OGD/R+DMSO+CCT groups using the densitometric analyses of western blot, in which the bar charts show the quantitative data (normalized by β-tubulin) for each protein relative to the control group (assigned a value of 1), data are represented as the mean±SD of three independent experiments, one-way ANOVA is used, *p<0.05, **p<0.01, ***p<0.001 vs. control group, #p<0.05, ##p<0.01, ###p<0.001 vs. OGD/R+DMSO group, $p<0.05, $$p<0.01, $$$p<0.001 vs. OGD/R+VAL group.

FIG. 5D shows quantification of the expression level of CHOP in the control, OGD/R+DMSO, OGD/R+VAL, OGD/R+VAL+CCT and OGD/R+DMSO+CCT groups using the densitometric analyses of western blot, in which the bar charts show the quantitative data (normalized by β-tubulin) for each protein relative to the control group (assigned a value of 1), data are represented as the mean±SD of three independent experiments, one-way ANOVA is used, *p<0.05, **p<0.01, ***p<0.001 vs. control group, #p<0.05, ##p<0.01, ###p<0.001 vs. OGD/R+DMSO group, $p<0.05, $$p<0.01, $$$p<0.001 vs. OGD/R+VAL group.

FIG. 5E shows quantification of the expression level of GRP78 in the control, OGD/R+DMSO, OGD/R+VAL, OGD/R+VAL+CCT and OGD/R+DMSO+CCT groups using the densitometric analyses of western blot, in which the bar charts show the quantitative data (normalized by β-tubulin) for each protein relative to the control group (assigned a value of 1), data are represented as the mean±SD of three independent experiments, one-way ANOVA is used, *p<0.05, **p<0.01, ***p<0.001 vs. control group, #p<0.05, ##p<0.01, ###p<0.001 vs. OGD/R+DMSO group, $p<0.05, $$p<0.01, $$$p<0.001 vs. OGD/R+VAL group.

FIG. 5F shows quantification of the expression level of c-caspase 3 in the control, OGD/R+DMSO, OGD/R+VAL, OGD/R+VAL+CCT and OGD/R+DMSO+CCT groups using the densitometric analyses of western blot, in which the bar charts show the quantitative data (normalized by β-tubulin) for each protein relative to the control group (assigned a value of 1), data are represented as the mean±SD of three independent experiments, one-way ANOVA is used, *p<0.05, **p<0.01, ***p<0.001 vs. control group, #p<0.05, ##p<0.01, ###p<0.001 vs. OGD/R+DMSO group, $p<0.05, $$p<0.01, $$$p<0.001 vs. OGD/R+VAL group.

FIG. 5G shows quantification of the expression level of bax in the control, OGD/R+DMSO, OGD/R+VAL, OGD/R+VAL+CCT and OGD/R+DMSO+CCT groups using the densitometric analyses of western blot, in which the bar charts show the quantitative data (normalized by β-tubulin) for each protein relative to the control group (assigned a value of 1), data are represented as the mean±SD of three independent experiments, one-way ANOVA is used, *p<0.05, **p<0.01, ***p<0.001 vs. control group, #p<0.05, ##p<0.01, ###p<0.001 vs. OGD/R+DMSO group, $p<0.05, $$p<0.01, $$$p<0.001 vs. OGD/R+VAL group.

FIG. 5H shows quantification of the expression level of bcl-2 in the control, OGD/R+DMSO, OGD/R+VAL, OGD/R+VAL+CCT and OGD/R+DMSO+CCT groups using the densitometric analyses of western blot, in which the bar charts show the quantitative data (normalized by β-tubulin) for each protein relative to the control group (assigned a value of 1), data are represented as the mean±SD of three independent experiments, one-way ANOVA is used, *p<0.05, **p<0.01, ***p<0.001 vs. control group, #p<0.05, ##p<0.01, ###p<0.001 vs. OGD/R+DMSO group, $p<0.05, $$p<0.01, $$$p<0.001 vs. OGD/R+VAL group.

### DETAILED DESCRIPTION OF THE PREFERRED EMBODIMENT

In order to make the present invention clearer, the present invention will be further described in detail as below in combination with embodiments. It should be understood that the specific embodiments described herein are merely illustrative of the present invention and are not intended to limit the present invention, that is to say, the described embodiments herein are only a part of the embodiments of the present invention, but not all the embodiments. Based on the embodiments of the present invention, all other embodiments obtained by those skilled in the art without creative work will fall into the protection scope of the present invention. Unless otherwise defined, the terms used below are consistent with those understood by professionals in the field. Unless otherwise specified, the raw materials, reagents or equipment mentioned herein may be purchased from the market or obtained through known methods.

Materials and Methods:

Through animal ethics, cell culture, animal selection, reagent and antibody information, anterior chamber compression rat model is established, R28 cell OGD/R (oxygen-glucose deprivation/reoxygenation) model is established; HE (hematoxylin-eosin) staining, apoptosis by TUNEL (TdT-mediated dUTP Nick-End Labeling) detection, cell viability by CCK8 (Cell Counting Kit-8), Western blot assay, Annexin V-FITC/PI flow cytometry, PI/Hoechest staining and immunofluorescence assay are performed.

### First Embodiment

Valdecoxib protects R28 from OGD/R injury by inhibiting apoptosis in vitro.

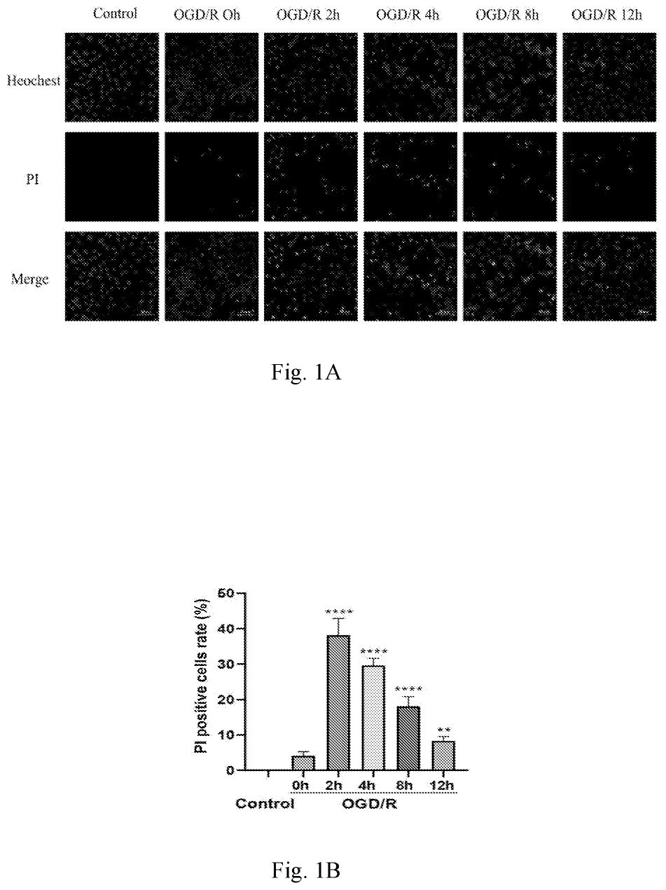

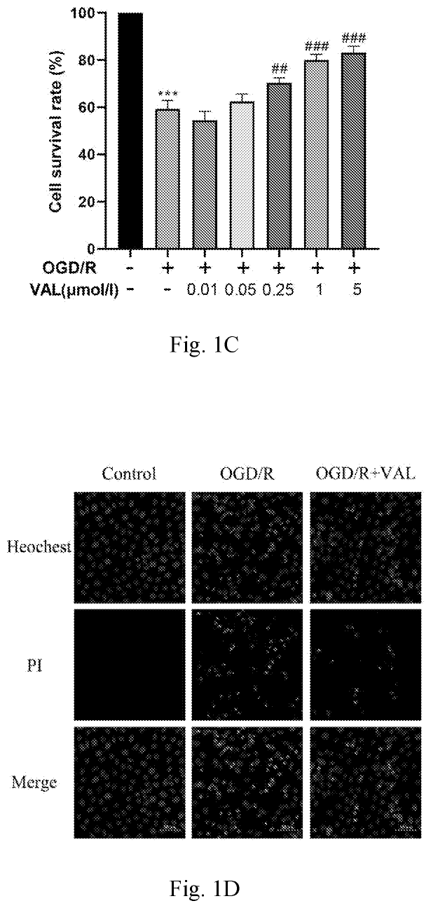

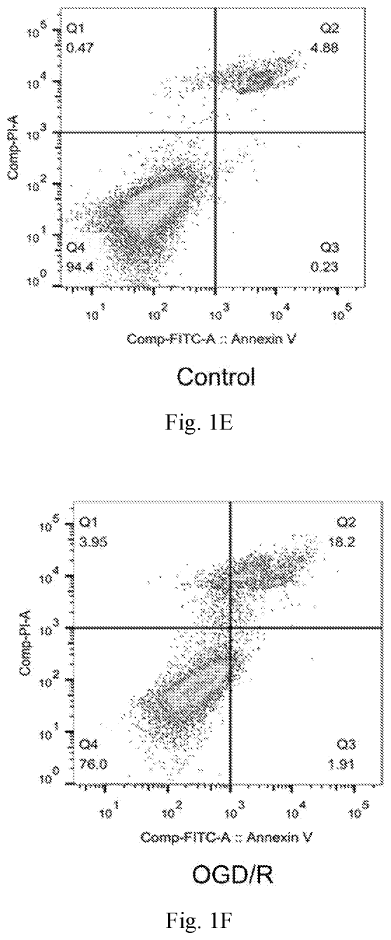

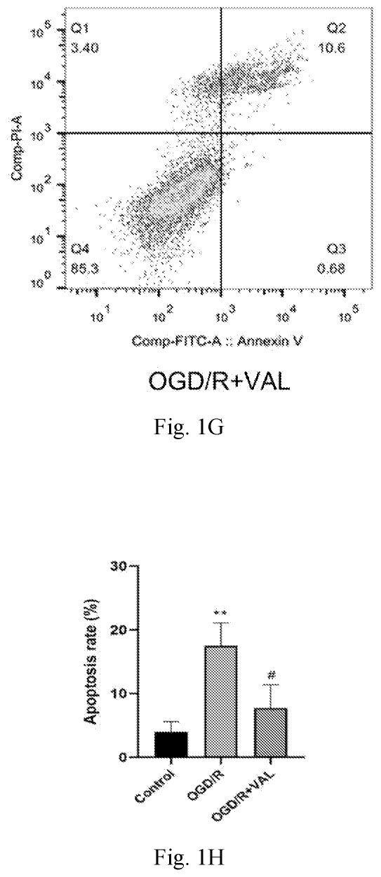

The cell death rate at multiple time points with the OGD/R model is detected using PI staining. PI-positive cells are identified as dead cells. The proportion of PI-positive cells at multiple time points are calculated. The highest PI-positive cell rate is observed at 2 hours after OGD/R, as shown in FIGS. 1A and 1B. Next, to test whether the valdecoxib is able to protect the R28 from OGD/R-mediated cell death, CCK-8 is performed to identify the valdecoxib's effects on the R28 cells in the OGD/R model at different concentrations at 2 h post-OGD/R. The valdecoxib treatment significantly elevates the cell survival rate at the concentration of 1 μmol/L and 5 μmol/L as shown in FIG. 1C. PI staining is further used to determine the valdecoxib's protective effect, as shown in FIG. 1D. To identify whether the valdecoxib's effect involved an anti-apoptosis mechanism, FCM (flow cytometry) is performed, and a further analysis indicates both that OGD/R induced cell apoptosis and the valdecoxib decreases the cell apoptosis rate, as shown in FIGS. 1E-1H. Based on these results, it is concluded that valdecoxib protects R28 from OGD/R injury by inhibiting apoptosis.

### Second Embodiment

VAL protects the retina from IRI (ischemia-reperfusion injury) by inhibiting apoptosis.

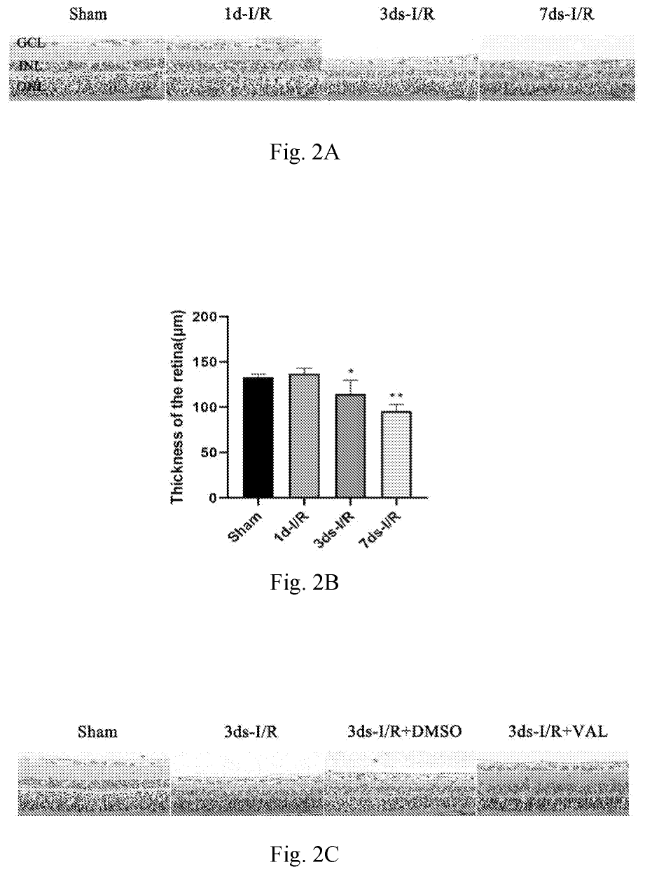

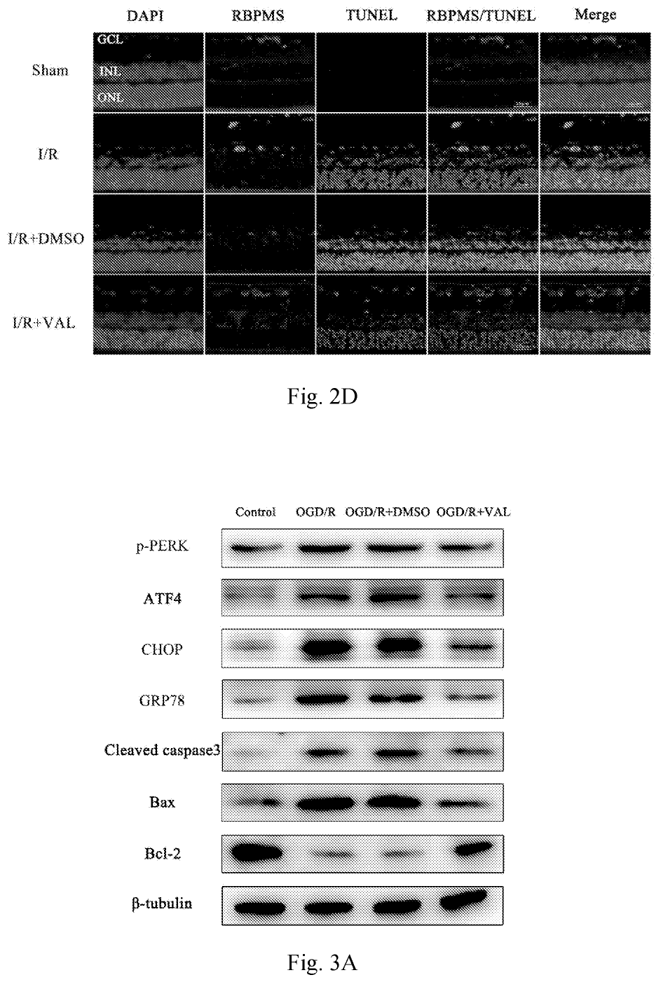

In order to evaluate the potential therapeutic prospect of VAL for saving RGCs (retinal ganglion cells) in glaucoma retinal injury, the glaucoma retinal IR is simulated by performing an aHIOP (acute high intraocular pressure) model on SD (Sprague-Dawley) rats. The rats are sacrificed and their eyeballs are removed at 1, 3 or 7 days post-IRI. HE staining is performed to detect the morphological changes in the retina. It is discovered that, compared to the control group, the retinas from 3 and 7 days post-IRI are markedly thinner. Additionally, lost RCGs or their disordered arrangement is observed at 3 and 7 days after IRI when compared with the control retina, as shown in FIGS. 2A and 2B. Next, the valdecoxib's effect on the RGCs in the FR model by HE staining and immunofluorescence. The valdecoxib significantly increases the retinal thickness and RGCs' survival rate at 3 days post-injury, as shown in FIG. 2C. A TUNEL assay is performed to clarify whether valdecoxib's protective effect involves the anti-apoptosis mechanism. RBPMS and TUNEL are used to label the RGCs and apoptotic cells of the retina, respectively. It is demonstrated that the TUNEL-positive RGCs increased after FR and are reduced after the valdecoxib treatment compared to the FR group, as shown in FIG. 2D. The above results indicate that valdecoxib is able to attenuate IRI-induced RGC loss and retina injury by inhibiting apoptosis.

### Third Embodiment

VAL inhibits R28 apoptosis by alleviating PERK-ATF4-CHOP pathway-mediated ER stress.

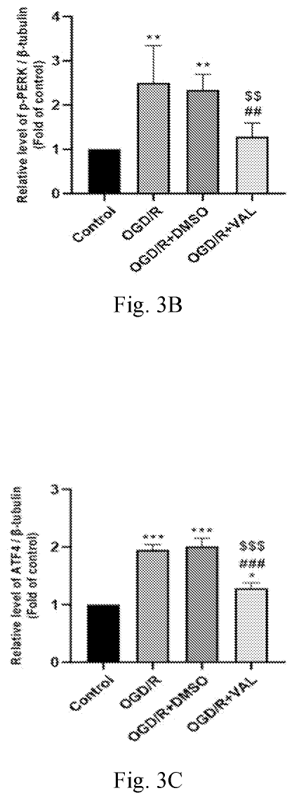

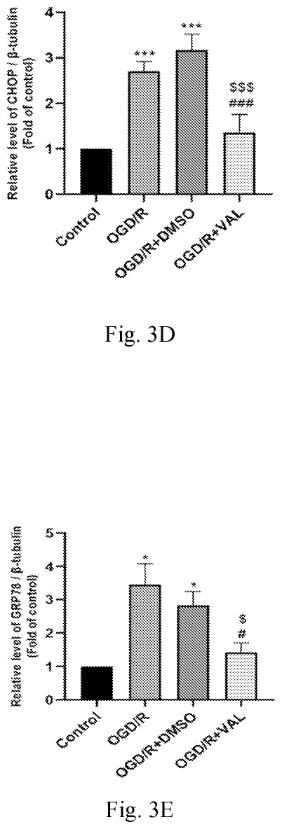

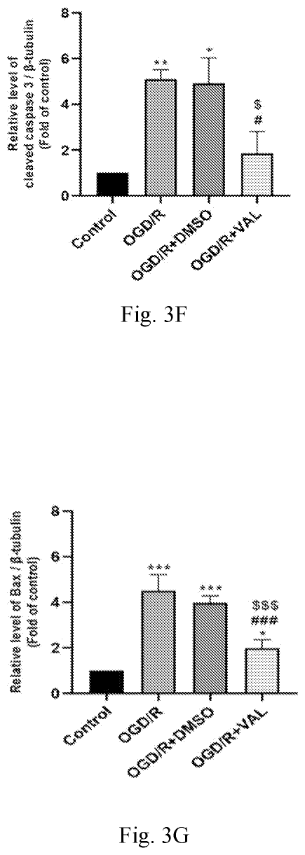

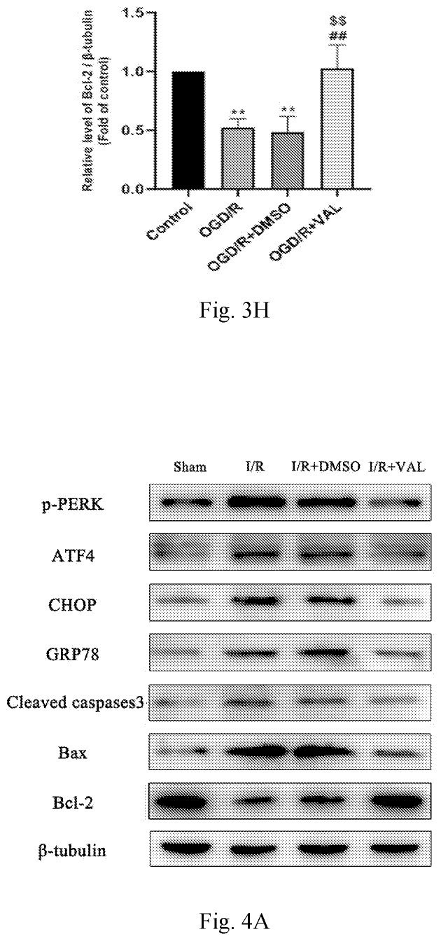

Previous studies show that OGD/R induces ER stress and increases activating transcription factor (ATF4) and CHOP protein levels. It is further examined whether the protein kinase RNA-like endoplasmic reticulum kinase (PERK)-ATF4-CHOP pathway is inhibited in the OGD/R model after valdecoxib pretreatment. Western blotting shown in FIG. 3A and its densitometric analyses shown in FIGS. 3B to 3H demonstrate that the GRP78, p-PERK, CHOP and ATF4 protein levels are markedly upregulated in the OGD/R and OGD/R+DMSO groups compared to the control group. The valdecoxib decrease the expression of those proteins during OGD/R injury. In addition, compared to the control group, the OGD/R and OGD/R+DMSO groups significantly elevate the apoptosis-related proteins, including bax and cleaved caspase 3, while these proteins are decreased in the valdecoxib pretreatment group, as shown in FIG. 3A. The expression of the anti-apoptosis protein bcl-2 is the opposite to that of the bax and cleaved caspase 3 in each group. The expression levels of the pro-apoptosis proteins bax and cleaved caspase 3 are upregulated, along with the activation of the PERK-ATF4-CHOP pathway, and decreased together with the inhibition of the PERK-ATF4-CHOP pathway. These data demonstrate that valdecoxib may inhibit R28 cells' apoptosis by alleviating PERK-ATF4-CHOP pathway-mediated ER stress.

### Fourth Embodiment

VAL protects the retina from IRI-mediated apoptosis by alleviating PERK-ATF4-CHOP pathway-mediated ER stress.

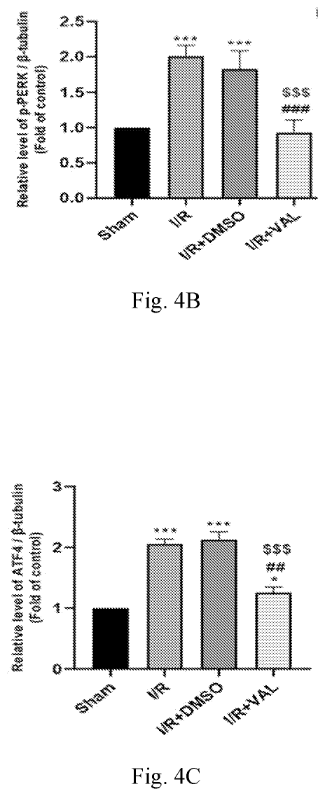

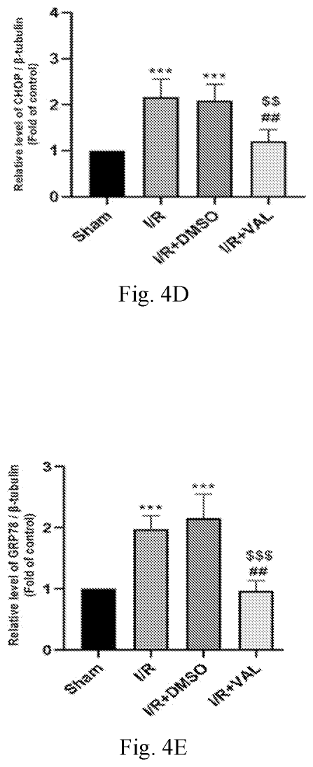

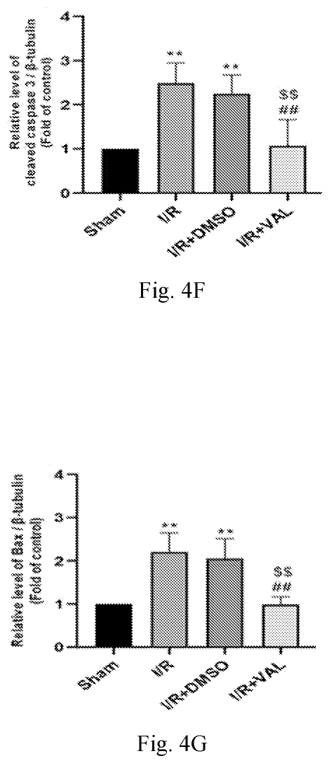

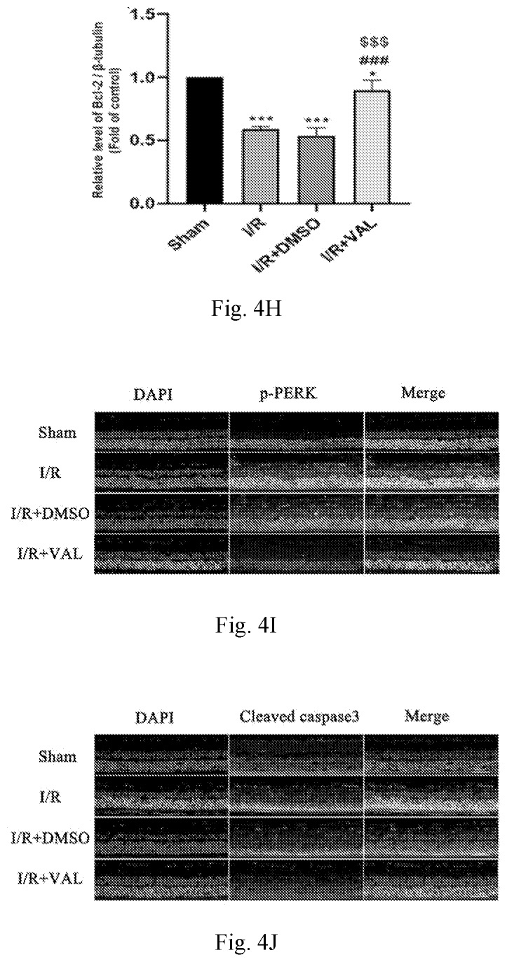

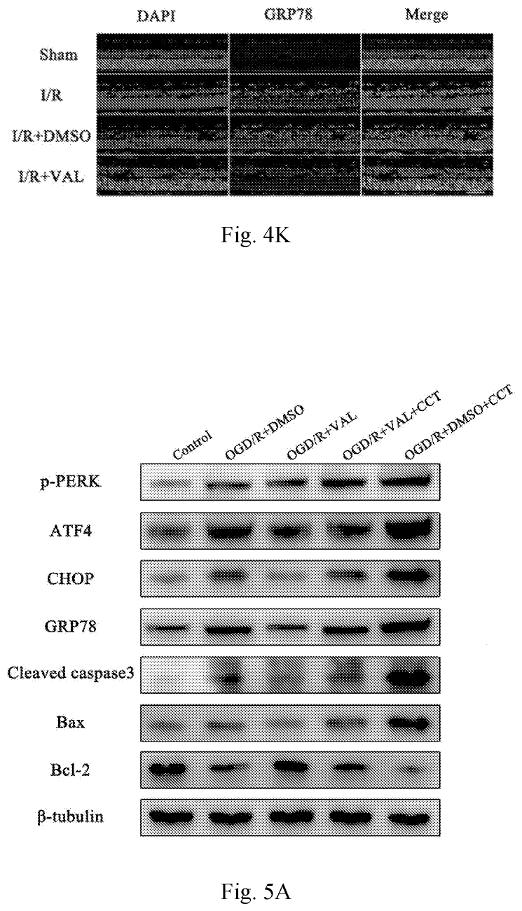

After the valdecoxib's effect on the PERK-ATF4-CHOP pathway and the cell apoptosis in the OGD/R model are demonstrated, it is examined whether valdecoxib exerts a protective effect on the retina in IRI through similar mechanism. Retina lysates are subjected to Western blot analysis, which demonstrates that the expression levels of p-PERK, ATF4 and CHOP are increased in the I/R and I/R+DMSO groups compared to the control retina, while these proteins are reduced in the retinas pretreated with valdecoxib during the I/R. The expression of the ER stress protein GRP78 is consistent with the PERK-ATF4-CHOP pathway proteins, as shown in FIGS. 4A to 4H. The expression levels of the apoptosis-related proteins are examined in the control, I/R, I/R+DMSO and I/R+valdecoxib groups. As shown in FIG. 4A, when using Western blotting, compared to the control, there is an increased expression of bax and cleaved caspase 3 in the retinas of the FR and I/R+DMSO groups. The valdecoxib decreases the expression of these proteins in the retina during I/R injury. The expression of the anti-apoptosis protein bcl-2 is the opposite of that of the bax and cleaved caspase 3 in each group, as shown in FIGS. 4A to 4H. The expression levels of p-PERK, cleaved caspase 3 and GRP78 in RGCs are evaluated by immunofluorescence. The results obtained in the immunofluorescence analyses are in accordance with the corresponding Western bolt results, as shown in FIGS. 4I to 4K. These results support the conclusion that valdecoxib protects the retina from IRI-mediated apoptosis by alleviating PERK-ATF4-CHOP pathway-mediated ER stress.

### Fifth Embodiment

CCT020312 reverses valdecoxib's anti-apoptosis effect by activating PERK-ATF4-CHOP pathway-mediated ER stress in vitro.

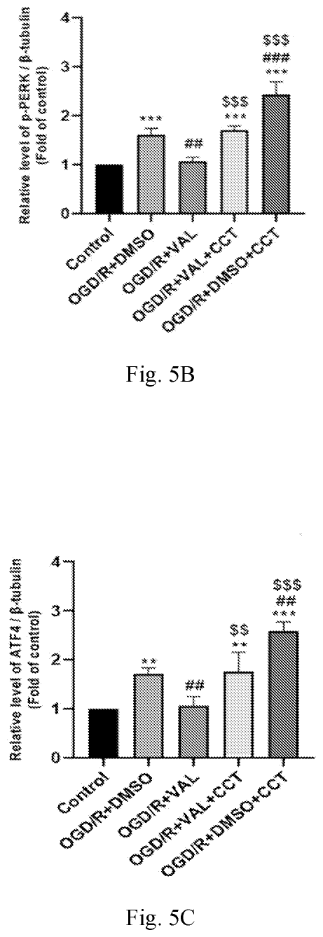

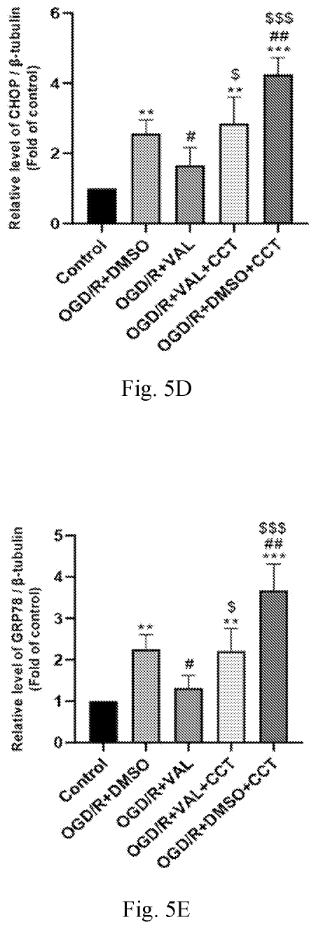

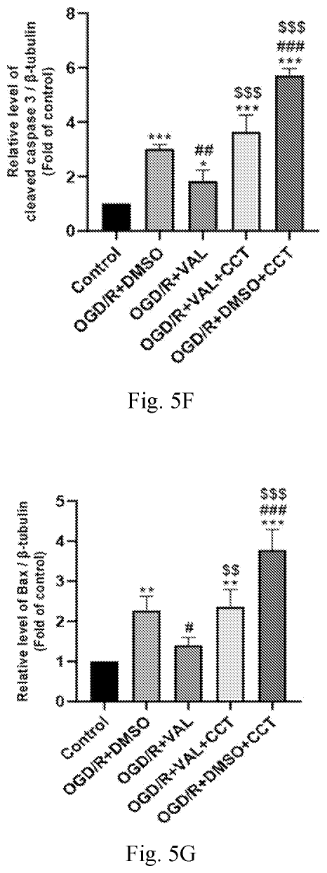

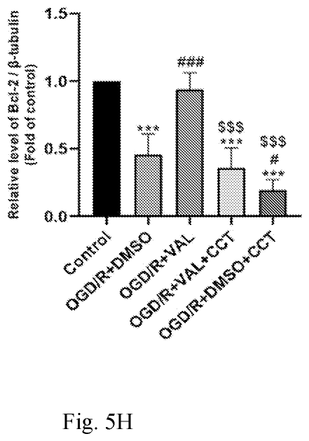

Recent studies have identified that CCT020312, as a selective activator of PERK, is able to activate the PERK-ATF4-CHOP signaling pathway. It is examined whether activating the PERK-ATF4-CHOP pathway by using CCT020312 is able to reverse the anti-apoptosis effect of valdecoxib in the OGD/R model. The R28 cells are pretreated with different concentrations of CCT020312 and no significant cell death is observed. The R28 is pretreated with valdecoxib prior to CCT020312 administration, after which it is subjected to the OGD/R model. The cell lysates collected from each group are subjected to a Western blot analysis of various markers of ER stress, apoptosis and the PERK-ATF4-CHOP pathway. A densitometric analysis confirms that the valdecoxib significantly reduces the OGD/R-induced GRP78, p-PERK, ATF4 and CHOP, as well as the expression of apoptosis-related proteins, including bax and cleaved caspase 3. The CCT020312 reverses the valdecoxib's effects, increasing the expression of markers of ER stress and the PERK-ATF4-CHOP pathway, as shown in FIGS. 5A to 5H. Next, it is determined whether the activation of the PERK-ATF4-CHOP induced by the CCT020312 increases the expression of the apoptosis-related proteins. The Western blot results show that the expression levels of the pro-apoptosis proteins, including bax and cleaved caspase 3, are consistent with those of the PERK-ATF4-CHOP pathway proteins in the CCT020312 pre-treated group, as shown in FIGS. 5A to 5H. These studies support the hypothesis that CCT020312 reverses valdecoxib's anti-apoptosis effect by activating PERK-ATF4-CHOP pathway-mediated ER stress.

Result Analysis

It is examined whether VAL has a protective effect in glaucoma cells and animal models, and whether the underlying molecular mechanism is related to the ERS. In the study, it is found that VAL increases cell viability in the OGD/R model and reverses the results of acute ocular hypertension retinal injury. The acute intraocular pressure elevation model (aHIOP) of rat eyeball leads to morphological changes of retina. In the glaucoma model, the ERS and apoptosis are alleviated after VAL administration, which further suggests that VAL is able to inhibit ER stress-mediated apoptosis to prevent and treat glaucoma injury. The activation of PERK-ATF4-CHOP pathway increases the expressions of pro-apoptosis proteins and decreases the expressions of anti-apoptosis proteins in the glaucoma retina and OGD/R R28 cells of rats. VAL avoids glaucoma injury by inhibiting ER stress-mediated apoptosis induced by the PERK-ATF4-CHOP pathway.

It is determined that VAL has a protective effect on glaucoma models in vivo and in vitro. The morphological changes of RGCs loss and retinal thinning are observed at three and seven days after retinal IR. However, intravitreal injection of VAL reverses the morphological changes. Moreover, the expressions of pro-apoptosis proteins are increased in the I/R group, but are decreased after VAL treatment. These results indicate that VAL is able to reduce IR-induced glaucoma injury by inhibiting RGC apoptosis. Similar results are observed in cellular models of glaucoma. The OGD/R group induces cell death, and VAL is able to significantly improve the cell survival rate. High expressions of pro-apoptosis proteins are observed in the OGD/R group, but the expressions of pro-apoptosis proteins are inhibited in the VAL group. These studies show that induction of apoptosis is indeed a key feature of glaucoma pathology. RGC apoptosis is the ultimate common pathway in both human and experimental glaucoma models, which is consistent with numerous studies. Inhibition of apoptosis is able to restore glaucoma injury. VAL avoids glaucoma injury by inhibiting apoptosis. The present invention is the first study to determine the protective effect of VAL in glaucoma.

Previous studies have shown that increased ER is one of the reasons for the development of intraocular pressure and glaucoma. Reduced ERS is able to prevent ocular lesions in mouse models of glaucoma. However, most studies have focused on ERS in the trabecular reticulum of glaucoma. Little is known about the link between RGCs, ERS and underlying mechanisms. In this study, it is examined the expressions of marker proteins of ERS in the FR retina. It is proved that the expressions of phosphorylated PERK, ATF4, CHOP and GRP78 are increased in the FR retina and are decreased in the VAL treatment group, which indicates that the induction of PERK, ATF4 and CHOP is related to the marker protein GRP78 of ERS. Apoptosis-related proteins, including Bax, Bcl-2, and cleaved caspase3, are further measured. The results show that the activation of the PERK-ATF4-CHOP pathway is consistent with the expressions of pro-apoptosis proteins, and is contrary to the expressions of anti-apoptosis proteins. VAL inhibits the PERK-ATF4-CHOP pathway in advance to increase the expressions of the anti-apoptosis proteins. Similar results are obtained in cellular models of glaucoma. These studies have shown that the activation of the PERK-ATF4-CHOP pathway plays a key role in ER stress-mediated apoptosis of glaucoma models. VAL plays a protective role in the glaucoma model by inhibiting the PERK-ATF4-CHOP pathway to induce ER stress-mediated apoptosis. In addition, in the OGD/R model, CCT020312 administration abolishes valdecoxib's protective effect, activates the expressions of p-PERK, ATF4 and CHOP, and aggravates the ER stress-mediated apoptosis in the OGD/R model, indicating that the inhibition of the PERK-ATF4-CHOP pathway is required for valdecoxib's protective effect on glaucoma.

In the study, it is shown that VAL exerts a protective effect on the glaucoma model by inhibiting ERS-induced apoptosis. Targeted PERK-ATF4-CHOP pathway is considered to be a potential mechanism for the protective effect of VAL. This is the first study to explore valdecoxib's function in glaucoma models, providing a potential treatment for glaucoma. These results provide clues as to how to better understand the mechanism of ER stress in glaucoma.

In conclusion, the study shows that the PERK-ATF4-CHOP pathway plays a significant pathological role in glaucomatous damage. Valdecoxib protects against glaucomatous injury by inhibiting endoplasmic reticulum stress-induced apoptosis via the inhibition of the PERK-ATF4-CHOP pathway. These findings suggest a promising role for valdecoxib therapy in protecting individuals from glaucoma.

The above is only the specific implementation mode of the present invention, but the protection scope of the present invention is not limited to this. Any variation or substitution that is able to be easily thought by those skilled in the art within the technical scope disclosed by the present invention shall be covered by the protection scope of the present invention.

What is claimed is:

1. A preparation method of a medication associated with a PERK-ATF4-CHOP signaling pathway for preventing and treating glaucoma, the preparation method comprising a step of administering VAL (Valdecoxib).

2. The preparation method according to claim 1, wherein the VAL is a regulating agent for the PERK-ATF4-CHOP signaling pathway to prevent and treat glaucoma.

3. The preparation method according to claim 2, wherein the VAL is able to inhibit ERS (endoplasmic reticulum stress) by regulating the PERK-ATF4-CHOP signaling pathway, so as to prevent and treat glaucoma.

4. A medication composition for preventing and treating glaucoma, comprising VAL (Valdecoxib).

5. The medication composition according to claim 4, further comprising a pharmaceutically acceptable carrier.
